(19) United States
(12) Patent Application Publication
(10) Pub. No.: US 2015/0265805 A1
(43) Pub. Date: Sep. 24, 2015

(54) Catheter with Gills

(71) Applicants: Ahmad D. Vakili, Tullahoma, TN (US); Dinesh Gupta, Tullahoma, TN (US)

(72) Inventors: Ahmad D. Vakili, Tullahoma, TN (US); Dinesh Gupta, Tullahoma, TN (US)

(21) Appl. No.: 14/628,239

(22) Filed: Feb. 21, 2015

Related U.S. Application Data
(60) Provisional application No. 61/966,315, filed on Feb. 21, 2014.

(51) Int. Cl. A61M 25/00; A61M 5/00

(52) U.S. Cl. CPC A61M 25/0075; A61M 5/007

(57) ABSTRACT

A guide catheter has an inlet orifice that allows blood to enter an interior passage of the catheter. A valve is positioned next to the inlet orifice that selectively opens and closes the inlet orifice. The catheter includes a dye passage that transports dye from a proximal end of the catheter to a dye outlet orifice. The dye passage may be formed in a unitary tubular member, a partitioned tubular member or in the clearances of a double-walled catheter. A valve may also be positioned next to the dye outlet orifices that selectively opens and closes the orifices. The valves are preferably kidney-shaped expandable bladders or pressure valves. A capillary tube connected to the expandable bladder functions to inflate and deflate the expandable bladder. The expandable bladders and capillary tubes are incorporated into the wall of the catheter.

### CROSS-REFERENCES TO RELATED APPLICATIONS

This application claims priority from co-pending U.S. Provisional Patent Application Ser. No. 61/966,315 filed Feb. 21, 2014, entitled “Coronary Guide Catheter with Gills” which is hereby incorporated by reference.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

Arterial catheters are designed and constructed to help cardiologists gain access to heart arteries for diagnostics and other purposes. Catheters are generally constructed of a thin walled linear tubing having an inlet fitting and a slightly curved tip region or hooked end. There are slight, variations in the designs of the most frequently used heart catheters that provide functional differences.

Some catheters are also used to inject tracer dye into specific locations in heart arteries to allow imaging and visualization of the blood flow in the regions fed by the artery where the catheter is positioned. The dye is injected into an inlet fitting at the end of the catheter which is outside of the patient body, and is expected to exit from the tip of the other end of the catheter into the artery. Ideally, the smaller the volume of dye injected into the body the better it is for the patient. This entails positioning the catheter inside narrow arteries, dose to potential blockages. In such cases, the catheter may restrict or fully block the flow of blood into the region fed by the artery into which it is inserted. When blood flow is restricted or shut of to a region of the heart by a catheter for a while this may result in a catheter induced heart myocardial ischemia, which is not desirable. The catheter is more likely to block or limit blood flow when used under certain situation such as in hard to reach narrow arteries. To allow blood to enter the catheter from the aorta and be ducted through the catheter to the tip of the catheter and hence to the narrow regions of the affected artery, one or two holes are typically built into many types of catheters approximately two inches before the tip. These small holes allow the patient's blood to enter the catheter to supply the artery while it is being accessed. This reduces the occurrence of situations where the lack of blood flow to regions supplied by the artery may lead to myocardial ischemia, which is induced by the catheter obstructing blood flow. Unfortunately, these blood inlet holes also result in tracer dye leaving the catheter through the holes into the main blood stream, which affects the diagnostic accuracy of the procedure by reducing contrast, and, thus, is not desirable.

One particular type of catheter, which typically has two holes located near the tip region of the catheter, is known as a Guide Catheter with Side Holes (GCSH). The holes' locations and size are designed to allow sufficient blood to enter the catheter from the aorta and force the blood through the holes into the catheter and out of the end tip into the narrow arteries. There are a few fundamentally undesirable disadvantages associated with wide use of the GCSH. The first disadvantage is that the solid catheter must he removed and replaced with the GCSH. This increases the time required for the operation and potentially increases complications due to the frequent removal and insertion of the different catheters. In addition, dye is injected periodically to position the catheter tip and locate the stents positions accurately. Some of the dye injected into the catheter leaks into the aorta and does not reach its intended target region of the heart. The leaking of dye limits the contrast and resolution of the images and potentially the accuracy of diagnostics. To compensate for this lack of contrast/resolution, the surgeon typically injects more dye at increased frequency into the catheter to make up for the diffused dye and lack of image resolution. Unfortunately, this leads to injecting the patient with more dye than ideally would be required. The increased dose of dye exposes the patient to greater risk of Nephrotoxicity. Equivalent situations can occur during the remedial phase of an operation when stents are inserted into is constricted artery, typically narrowed due to plaque built up.

Therefore, what is needed is an improved, arterial catheter that eliminates or reduces the need for excess dye injection and frequency of catheter removal.

### BRIEF SUMMARY OF THE INVENTION

An embodiment of the present invention is directed toward a catheter that has an inlet orifice that allows blood to enter an interior passage of the catheter. A valve is positioned next to the inlet orifice that selectively opens and closes the inlet orifice. The catheter includes as dye passage that transports dye from is proximal end of the catheter to a dye outlet orifice. A valve is also positioned next to the dye outlet orifice that selectively opens and closes the dye outlet orifice. The valves are preferably kidney-shaped expandable bladders. A capillary tube connected to the expandable bladder functions to inflate and deflate the expandable bladder. The expandable bladders and capillary tubes are incorporated into the wall of the catheter.

Another embodiment of the present invention is directed toward a catheter that includes an interior tubular member having a main passage formed therein. An inlet hole allows blood to enter the main passage in the interior tubular member and an exit hole allows blood to exit the main passage in the interior tubular member. An exterior tubular member surrounds the interior tubular member such that an auxiliary passage is formed between an exterior surface of the interior tubular member and an interior surface of the exterior tubular member. An auxiliary inlet allows dye to enter the auxiliary passage and an auxiliary outlet allows dye to exit the auxiliary passage. A pressure or bladder valve selectively opens and closes the auxiliary inlet or outlet.

Yet another embodiment of the present invention is directed toward a catheter that includes a unitary tubular member having a main passage and an auxiliary passage formed in an interior of the unitary tubular member. A main inlet orifice allows blood to enter the main passage and a main outlet orifice that allows blood to exit the main passage. An auxiliary inlet allows dye to enter the auxiliary passage and an auxiliary outlet orifice allows dye to be expelled from the auxiliary passage. A valve is positioned next to the auxiliary inlet or outlet orifice that selectively opens and closes the auxiliary inlet or outlet orifice. A valve is also positioned next to the main inlet orifice that selectively opens and closes the main inlet orifice. The unitary tubular member may include, a second auxiliary passage formed therein.

### BRIEF DESCRIPTION OF THE SEVERAL VIEWS OF THE DRAWINGS

FIG. 1 is an illustration of a guide catheter with gills constructed in accordance with an embodiment of the present invention;

FIG. 2(A) is an illustration of a double-walled guide catheter constructed in accordance with another embodiment of the present invention;

FIG. 2(B) is a close up illustration of the tip region of the double-walled guide catheter of FIG. 2(A);

FIG. 3(A) is an illustration of a partial double-walled guide catheter constructed in accordance with an embodiment of the present invention;

FIG. 3(B) is a close up illustration of the tip region of the partial double-walled guide catheter of FIG. 3(A);

FIGS. 4(A-C) are illustrations of guide cross sections having multiple lumens in accordance with another embodiment of the present invention;

FIGS. 5(A) and 5(B) are illustrations of a guide catheter valve constructed in accordance with the present invention.

### DETAILED DESCRIPTION OF THE INVENTION

The present invention is directed toward a new family of guide catheters, such as coronary guide catheters, designed to improve upon existing guide catheters by making them more versatile and multipurpose while improving their safety and efficiency of use. The invention uses designs that employ different arrangement mechanisms to integrate orifices that are used to allow uninterrupted blood flow into the artery being accessed. The invention is composed a number of specific catheter designs that accommodate blood flow to the artery from each catheter's tip region while the catheters are in service.

The present guide catheters with gills have one or more of the following improvements or innovations integrated into the catheter: modified orifices, orifice regions, and or dye injection passages with features that enable the opening or closing of the orifices. These innovations are directed toward delivering the tracer dye at the very tip of the catheter guide with gills at all times during their use. Furthermore, these catheters have certain integrated functional controls designed to open or close the orifices to minimize dye delivery to unintended locations such as larger open arteries. These are designed such that the various controls or valves can open or close in a short time, and be controlled manually or via microprocessor controlled interfaces, per command of the surgeon or their team. These catheters are generically referred to as “Guide Catheters with Gills” and individually described below.

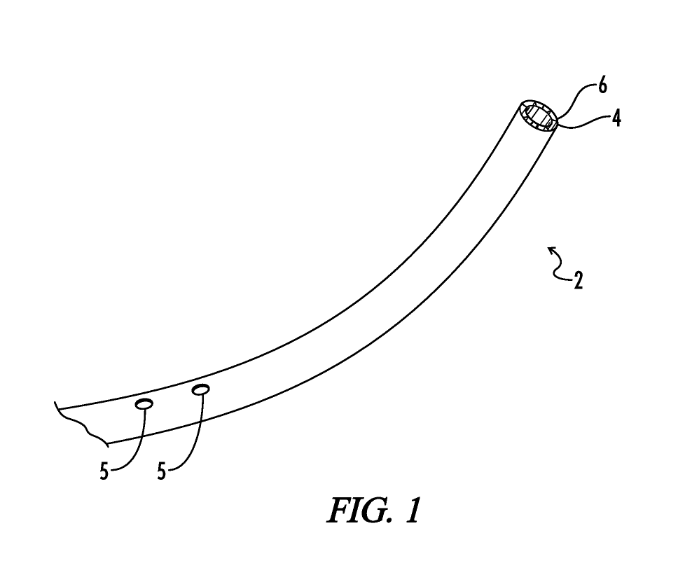

Referring Now to FIG. 1, an illustration of a guide catheter with gills constructed in accordance with an embodiment of the present invention is shown. Like an existing guide catheter, the guide catheter of FIG. 1 has side holes 5, or gills, that allow blood to enter the interior of the catheter 2. The guide catheter 2 also has specially designed dye expelling orifices 4 with associated hydraulically elastic and expandable micro bladders 6, such as planar inflatable bladders, integrated into each dye orifice 4 of the guide catheter 2. The inflatable micro bladders 6 are connected via one or more passages or capillary tubes, as discussed in more detail below, to dedicated proximal fittings outside of the patient near the beginning of the guide catheter 2. The proximal fitting facilitates the mechanism for connecting an especially designed liquid reservoir to fill and expand the expandable bladder 6 discs and close the orifices 4 to block dye flow out of the orifices 4. Thus, the bladders 6 function as a valve that opens during the short time that the dye is being injected into the artery.

In the especially preferred embodiment shown in FIG. 1, the guide catheter 2 employs a modified contoured orifice 4 that is shaped like a kidney and includes an elastic, expandable, micro-bladder pouch 6 to close the contoured kidney-shaped openings 4 in the end of the catheter 2 when needed. Bladder valves 6 are also provided for the blood entry orifices 5 to control the blood flow into the catheter and prevent dye from escaping from the catheter as described in more detail below. The supply tubing and expandable bladders 6 are preferably designed so they are integrated into the walls and holes 4 and 5 of the catheter 2 to ensure no internal or external protrusions from the catheter prior or during their operation.

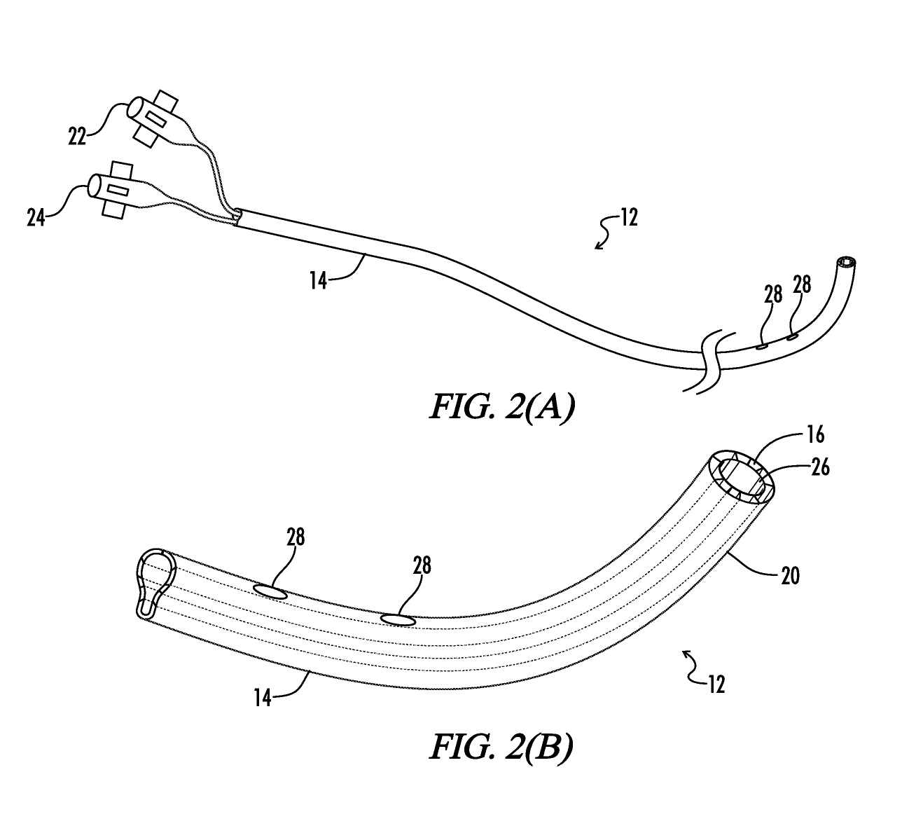

Referring now to FIGS. 2(A) and 2(B), illustrations of a double-walled guide catheter 12 constructed in accordance with embodiment of the present invention are shown. FIG. 2(B) is a close up illustration of the tip region of the double-walled guide catheter of FIG. 2(A). The guide catheter with gills 12 employs a double-walled guide 14, with clearances between the walls of the guide 14 tubing, along its full length, from its proximal end 18 to its distal end 20. The guide tubing 14 may be manufactured either with internal will partitions along the length of the double-walled guide catheter that form passages between the interior and exterior walls of the catheter, or as a dual-passage catheter guide with separate interior passages, as described in more detail below, especially manufactured with voids that function as dye supply passages.

The double-walled guide catheter 14, shown in FIGS. 2(A) and(B), has clearances between the interior and exterior walls that form multiple micro-passages 16. At the proximal end 18 of the catheter there are two independent inlet fittings 22 and 24 integrated into the inlet end 18 of the catheter 12. The additional inlet provides the mechanism to supply the dye into the guide 14 through the wall passages into its distal end 20, or tip region. The two inlet fittings 22 and 24 are preferably color coded and identified as the main inline fitting 22 (e.g. yellow) which provides access to the interior 26 of the guide 14 and one auxiliary side fitting 24 (e.g. green) which functions as a dye supply inlet. Dye is injected via the proximal secondary (green) fitting 24 which is connected into the space between the inner and the outer walls of the guide 14 to the distal tip 20 of the catheter 12.

The region of the catheter tip end 20 is minimally but appropriately modified to incorporate exit openings 16 for the dye to be delivered out of the tip region of the catheter. This is precisely the desired location for dye delivery, while the blood supply is still able to flow into and through the center 26 of the catheter 12 from built-in peripheral orifices 28 in the walls of the guide 14. As discussed herein, all of the orifices 16 and 28 can be provided with valves to selectively control the flow of dye and blood through the guide catheter.

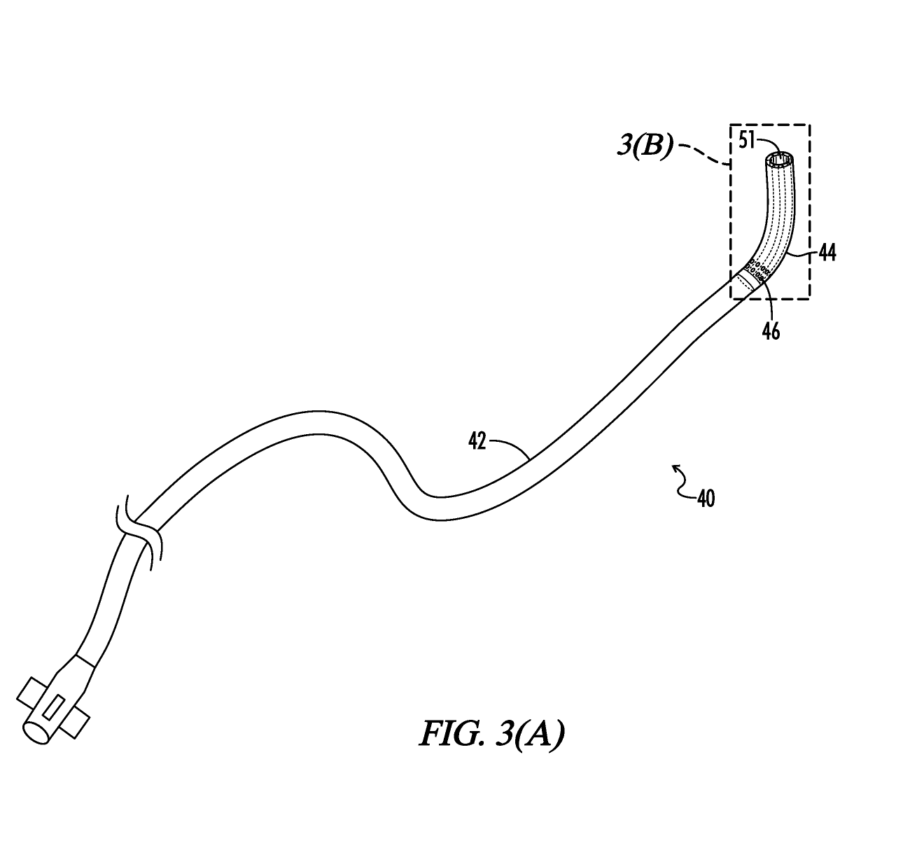

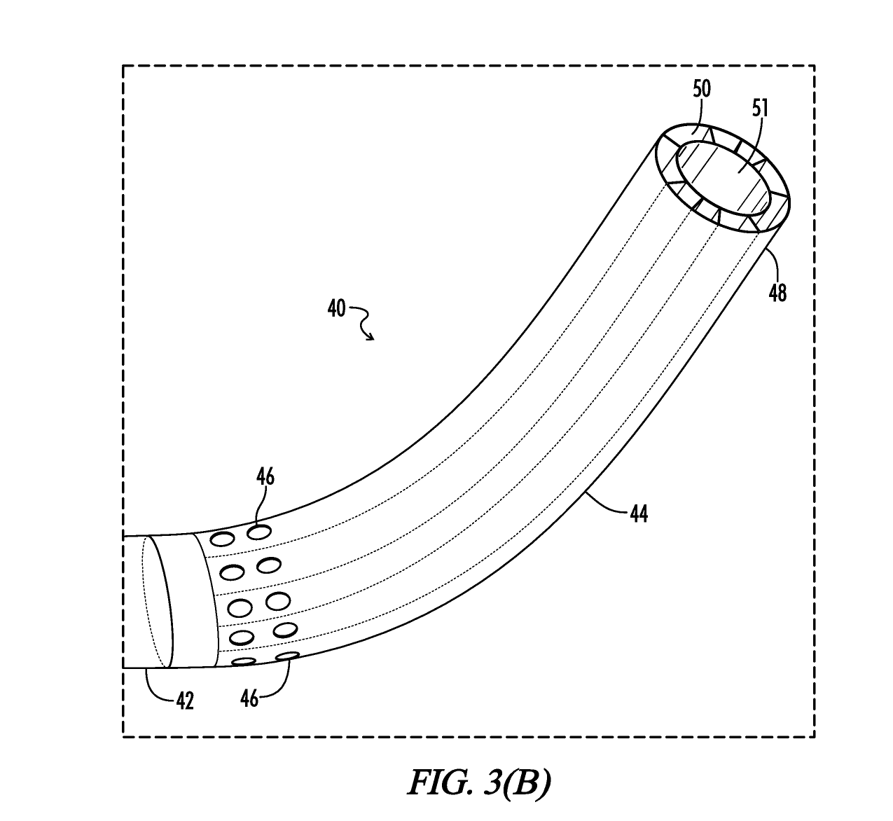

Referring now to FIGS. 3(A) and 3(B), illustrations of a partial double-walled catheter 40 constructed in accordance with an embodiment of the present invention are shown. FIG. 3(B) is a close up illustration of the tip region of the partial double-walled guide catheter of FIG. 3(A). The catheter 40 includes a limited length double-walled distal end 44. The blood supply to the end of the catheter 40 is facilitated from the peripheral gills or orifices 46 through the interior of the distal end 44 to the exit hole 51 at the distal tip 48 of the catheter via internal ducts. In addition, internal passages in the proximal end 42 are used to transport dye to the double-walled distal end 44. The double-walled distal end 44 has passages 50 formed between the interior and exterior walls that transport the dye received from the proximal end 42 to the distal tip 48 of the catheter 40. The location and shapes of both the blood entrance holes 46, blood exit hole 51 and dye orifices at the end of passages 50 are readily customizable depending upon the particular application. As discussed above, valves can be provided for the orifices 46 and 50 to selectively control the flow of blood and dye through the catheter 40 and prevent dye from escaping from the blood.

The catheter design of FIGS. 3(A) and 3(B) has a number of advantages over prior art guide catheter designs. These advantages include lower manufacturing costs, increased levels of customization and flexibility for children of different ages and adults, and retaining the current feel of existing catheters.

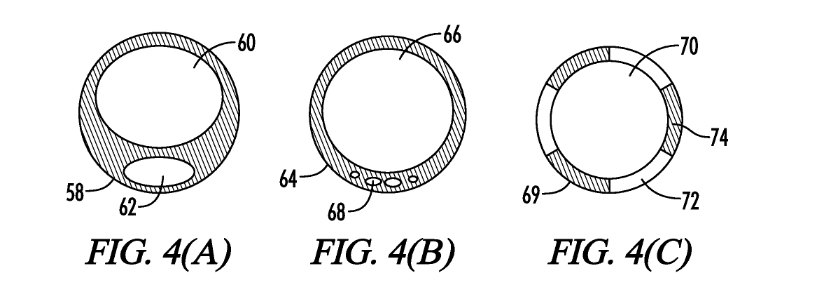

A variety of different multi-lumen guide cross sections can be used to implement the guide passages of the present invention. Referring now to FIGS. 4(A-C), illustrations of different guide cross sections having multiple lumens are shown. FIG. 4(A) depicts a multi-lumen guide cross section 58 that has a primary 60 and an auxiliary internal passage 62. FIG. 4(B) depicts a multi-lumen guide cross section 64 that has a single primary 66 and four auxiliary internal passages 68. FIG. 4(C) depicts a multi-lumen guide cross section 69 that has a single primary passage 70 and multiple auxiliary internal passages 72 formed by partitions 74 formed between an inner and outer wall of the guide 69. In view of the above, it will be readily apparent to those skilled in the art that a wide variety of different guide cross sections can be developed depending upon the desired application and characteristics.

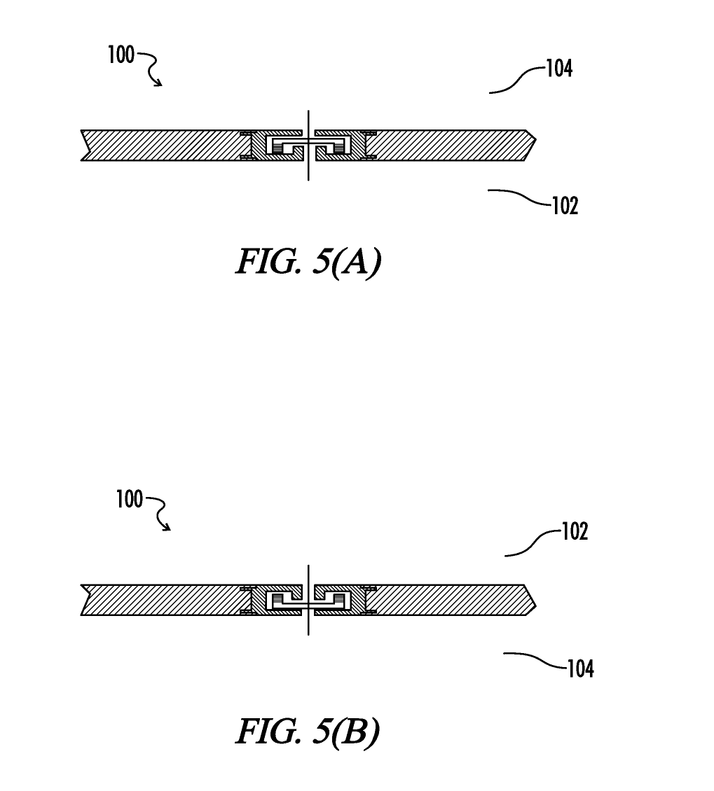

Referring now to FIGS. 5(A) and 5(B), illustrations of a catheter valve 100 for a catheter orifice in accordance with the present invention are shown. The guide catheter incorporates one or more miniature spring valves 100 to selectively open and close the orifices of the catheter. The valves are preferably constructed such that each valve 100 automatically opens or closes based on the pressure differential across it. The pressure differential is normally present due to the higher blood pressure in the aorta compared to the lower blood pressure in the arteries being accessed by the catheter guide used by the surgeon in the absence of dye being injected. The spring loaded mechanical membrane valve 100 is designed to function as a one-way valve. The valve is normally open and allows blood to flow from the outside of the catheter to the inside of the catheter. It is only during the short periods when surgeon or surgery team is injecting dye into the catheter that the internal pressure becomes higher than the external pressure and shuts the valve 100. The shutting of the valve in such a situation prevents the dye from leaving the catheter through the orifices into unintended regions of the body. Therefore, dye does not escape into the aorta and, hence, primarily exits out of the distal end tip of the catheter into the intended target regions.

The two modes of operation of the miniature guide valve 100 of the present invention are shown in FIGS. 5(A) and 5(B). As shown in FIG. 5(A), when dye is not being injected, the external pressure 104 is higher than the catheter internal pressure and the valve stays open and allows blood from the aorta to enter the catheter which flows into the artery. As shown in FIG. 5(B), when the pressure internal 102 to the catheter guide valve 100 is higher than the pressure outside 104 of the catheter valve, as occurs during dye injection, then the valve 100 closes and all of the dye is prevented from escaping through the valve and guided through the catheter distal end tip into the artery in support of imaging the potential problem area.

The above described new catheter designs have the advantage of both enabling the uninterrupted blood flow to the diseased heart arteries being evaluated for blockages and also minimizing the amount of tracer dye used for diagnosing blockages and/or to guide stent operations on patients. These catheters can be also used in connection with blood in a number of other applications including blood clot removal from the brain, lungs or other sensitive and critical organs. Other systems, methods, features, and advantages of the present disclosure will be or become apparent to those with skill in the art upon examination of the following drawings and detailed description. It is intended that all such additional systems, methods, features, and advantages be included within this description and be within the scope of the present disclosure. Thus, although there have been described particular embodiments of the present invention of a new and useful “Guide Catheter with Gills” herein, it is not intended that such references be construed as limitations upon the scope of this invention except as set forth in the following claims.

What is claimed is:

1. A catheter, said catheter comprising:
an inlet orifice that allows blood to enter an interior passage of said catheter; and
a valve positioned next to said inlet orifice that selectively opens and closes said inlet orifice.

2. The catheter of claim 1 further comprising a dye passage that transports dye from a proximal end of said catheter to a dye outlet orifice.

3. The catheter of claim 2 further comprising a valve positioned next to said dye outlet orifice that selectively opens and closes said dye outlet orifice.

4. The catheter of claim 1 wherein said valve further comprises an expandable bladder positioned near said inlet orifice.

5. The catheter of claim 4 further comprising a capillary tube connected to said expandable bladder that functions to inflate and deflate said expandable bladder.

6. The catheter of claim 4 further wherein said inlet orifice and said expandable bladder are kidney-shaped.

7. The catheter of claim 4 wherein said expandable bladder is incorporated into a wall of said catheter.

8. The catheter of claim 3 wherein said capillary tube is incorporated into a wall of said catheter.

9. A catheter, said catheter comprising:
an interior tubular member having a main passage formed therein;
an inlet hole that allows blood to enter said main passage in said interior tubular member;
an exit hole that allows blood to exit said main passage in said interior tubular member;
an exterior tubular member surrounding said interior tubular member such that an auxiliary passage is formed between an exterior surface of said interior tubular member and an interior surface of said exterior tubular member;
an auxiliary inlet that allows dye to enter said auxiliary passage; and
an auxiliary outlet that allows dye to exit said auxiliary passage.

10. The catheter of claim 9 further comprising a valve that selectively opens and closes said inlet hole.

11. The catheter of claim 1 where said valve further comprises an expandable bladder.

12. The catheter of claim 10 wherein said valve further comprises a pressure valve.

13. The catheter of claim 11 wherein said expandable bladder is kidney-shaped.

14. A catheter, said catheter comprising:
a unitary tubular member having a main passage and an auxiliary passage formed in an interior of said unitary tubular member;
a main inlet orifice that allows blood to enter said main passage;
a main outlet orifice that allows blood to exit said main passage;
an auxiliary inlet that allows dye to enter said auxiliary passage; and
an auxiliary outlet orifice that allows dye to be expelled from said auxiliary passage.

15. The catheter of claim 14 further comprising a valve positioned next to said main inlet orifice that selectively opens and closes said main inlet outlet orifice.

16. The catheter of claim 15 wherein said valve further comprises an expandable bladder.

17. The catheter of claim 15 wherein said valve further comprises a pressure valve.

18. The catheter of claim 14 further comprising a valve positioned next to said auxiliary outlet orifice that selectively opens and closes said auxiliary outlet orifice.

19. The catheter of claim 14 wherein said unitary tubular member further comprises a second auxiliary passage formed in said unitary tubular member.

20. The catheter of claim 14 wherein said main inlet orifice is kidney-shaped.
